(19) United States
(12) Patent Application Publication
(10) Pub. No.: US 2023/0117036 A1
(43) Pub. Date: Apr. 20, 2023

(54) INHALED AIR FILTRATION AND ENHANCED BREATHING DEVICE

(71) Applicant: Hussam Zebian, Milpitas, CA (US)

(72) Inventor: Hussam Zebian, Milpitas, CA (US)

(21) Appl. No.: 18/045,803

(22) Filed: Oct. 11, 2022

Related U.S. Application Data
(60) Provisional application No. 63/256,570, filed on Oct. 16, 2021.

(51) Int. Cl. A61M 16/04; A61M 16/10; A61M 16/12; A61M 16/08; A61M 16/00; A61M 16/16; A61M 16/20

(52) U.S. Cl. CPC A61M 16/049; A61M 16/106; A61M 16/12; A61M 16/0816; A61M 16/1075; A61M 16/0003; A61M 16/16; A61M 16/208; A61M 2202/0283; A61M 2205/056

(57) ABSTRACT

The present disclosure is directed to a plurality of air quality devices configured to be held in a user's mouth and configured to increase at least one of humidity and temperature and constitution of air inhaled. There are passive and active systems that allow for improved physical and lung performance.

### BACKGROUND

### Technical Field

The present disclosure relates to an enhanced filtration and air quality breathing device that enhances the quality of air inhaled into the lungs during mouth breathing.

### Description of the Related Art

The physical action of inhaling a deep breath requires a person to breathe in deeply, and somewhat slowly from the nose, to fill their lungs before exhaling. Others, while engaging in some form of hard physical activity and under physical duress, have gasped for air by breathing hard and quickly from the mouth. There has often been a common tradeoff between choosing quantity over quality, choosing to breathe from the mouth when we urgently need a large amount of air, instead of the nose. When needing that large amount of air, a person can only get a small amount of air from the nose, so naturally a person will switch to the mouth, which will quickly turn into shallow, fast, and heavy breaths.

Lungs require specific qualities of air to function properly. Specifically, lungs require high moisture content and high temperatures, otherwise, they will dry out and become less efficient. Measurements of air properties entering the lungs were conducted for people breathing in through the nose versus people breathing in through the mouth. Similarly, the properties of exhaled air were measured after air had left the body for both the nose and the mouth breathing subjects, leading to the conclusion that breathing through the nose can enhance the quality of the air because it purifies the air from pollutant, has a large surface area that heats up the air, increases the moisture of the breathed air, and produces a small amount of nitrous oxide (N2O).

Presently, there is no similar mechanism for the mouth. There are known similar devices that help patients who undergo a tracheostomy surgery breathe a better-quality air. However, those devices are bulky and require tubing and fixing, and even a mechanically assisted air flow inhale and exhale process, preventing any form of physical activity while using the device. Moreover, similar devices experience a much larger pressure loss and flow impedance because the device forces the air across a linear and constricted path through the filter, making it practically impossible to breathe fast through the device.

The devices in the medical field are made for small volume flowrates of air (5 liters/min), which prevents any form of elevated metabolic state of the user (up to 90 liters/min). There is no evidence of such a filter used by people who are conducting a normal life and have high physical activity, or in a harsh environmental condition, as opposed to tracheostomy patients who have no option but to breath from the trachea in a controlled environment. Therefore, there is a need to obtain a high-quality air like that of breathing through the nose, but with large volumes as though it was breathed through the mouth.

### BRIEF SUMMARY

The present disclosure is directed to a variety of enhanced air quality devices to be used by athletes or others to improve a quality of the air inhaled into their lungs. These devices significantly improve upon the quality of inhaled air into the lungs of the person wearing the enhanced air quality device during mouth breathing, particularly during physical activity or during harsh conditions of the ambient air or room air. The air quality devices include a portion inserted into the user's mouth. This is a comfortable, bitable portion to allow the user to keep this device in a steady position during use. The air quality devices are configured to enable breather through the user's mouth through an air filtration system to increase humidity and temperature of the air inhaled as compared to mouth breathing without these devices. The increase in humidity and temperature can improve the performance of the user.

In one embodiment, the device includes a bite plate and channel coupled to the bite plate. An attachment end is spaced from the bite plate by the channel. The attachment end includes an opening that is in fluid communication with the channel. The breathing device further includes an air filtration system that is removably coupled to the attachment end. Air flows through the air filtration system into the attachment end, then into the channel, and into a chamber that includes the bite plate in response to the user's inhale. The air filtration system is configured to be removable and replaceable after a filter has exceeded a predetermined useful life. For example, the filter may change colors once the filter has passed a threshold number of uses. Also as the type and intensity of the physical activity change, and or the ambient conditions change, the optimal thickness of the filter changes, and the user has the option to change the filter on the spot depending on the conditions he/she is being subjected to.

As an alternative to the previously introduced passive version, there are active versions of the air quality device. One active version includes a heater or heating device that is slidably received by the attachment end. The heater is coupled to a battery or other power supply by a cord or wire. The heater may be a plurality of interwoven wires that are surrounded by a frame. The wires may be arranged in an array or grid pattern that is positioned in the opening so that when air is inhaled, the air passes through the wires into the channel and into the chamber. Humidification capabilities may be added to that design as will be elaborated later.

In another active embodiment, the device includes a heater that is part of a moisture and heating system that is not directly positioned in front of the user's mount and instead is either extending from a side of the user's mouth or spaced from the user's mouth by tubing. The moisture and heating system includes an air intake valve that is coupled to a heater chamber, which heats up the air being inhaled. The heater chamber is coupled to a moisture chamber that includes a water atomizer or evaporator, which creates water vapor from a container of water resulting in moisturizing the inhaled air. The moisture chamber is coupled to the attachment end.

The active devices further include input from a power source for heating the elements and operating the moisture chamber. The power source is integrated with the device, in close proximity to the user's mouth or connected via a power cable place at a location away from the user's mouth.

In one embodiment, a bite plate is coupled to a duct attached the bite plate. A removable air exchange attachment is detachably coupled to the duct. A moisture tank and a heating element are both coupled to the air exchange attachment. The moisture tank includes a heating coil and an absorbent enclosure. In another example of the embodiment, a breathing device is configured to a user's mouth with a first end and a second end, with a duct coupled to the second end of the breathing device. A filtration system having a power source is coupled to the breathing device by the duct, where the filtration system includes a first one-way valve.

### BRIEF DESCRIPTION OF THE SEVERAL VIEWS OF THE DRAWINGS

For a better understanding of the embodiments, reference will now be made by way of example to the accompanying drawings. In the drawings, identical reference numbers identify similar elements or acts unless the context indicates otherwise. The sizes and relative portions of the elements in the drawings are not necessarily drawn to scale. For example, some of these elements may be enlarged and positioned to improve drawing legibility.

FIG. 1A is a perspective view of a mouthpiece of an inhaled air breathing device.

FIG. 1B is a perspective view of a filter attachment of the inhaled air breathing device of FIG. 1A.

FIG. 1C is a view of the mouthpiece of the inhaled air breathing device of FIG. 1A.

FIG. 1D is a side view of the mouthpiece of the inhaled air breathing device of FIG. 1A.

FIG. 2 is a perspective view of an alternative filter attachment in FIG. 1B.

FIG. 3A is a perspective view of an alternative embodiment of an inhaled air breathing device.

FIG. 3B is an exploded view of the device in FIG. 3A.

FIG. 4 is a cross-sectional view of an alternative embodiment of an inhaled air breathing device.

FIG. 5 is a perspective view of a portion of the device in FIG. 4.

FIG. 6 is an exploded view of an alternative embodiment of an inhaled air breathing device.

### DETAILED DESCRIPTION

In the ensuing description, certain specific details are set forth in order to provide a thorough understanding of various disclosed embodiments. However, one skilled in the relevant art will recognize that embodiments may be practiced without one or more of these specific details, or with other methods, components, materials, etc.

Reference throughout this specification to “one embodiment” or “an embodiment” means that a particular feature, structure or characteristic described in connection with the embodiment is included in at least one embodiment. Thus, the appearance of the phrases “in one embodiment” or “in the embodiment” in various places throughout this specification are not necessarily all referring to the same embodiment. Furthermore, the particular features, structures, or characteristics may be combined in any suitable manner in one or more embodiments.

The present disclosure is directed to an enhanced inhaled air quality and breathing device that can be passive or active, which will be explained in detail below. The breathing device enhances a quality of air inhaled by a user. The devices in this disclosure are directed to enhancing the quality of the air inhaled into lungs of the person wearing the device during mouth breathing, particularly during physical activity or during harsh or non-ideal conditions of ambient air or room air; air conditions are notorious for creating a very dry cold air. While cold and dry air is good for the body's cooling effect during a workout, it is very bad for the lungs. These devices may be modified for animals, especially those that are perform demanding physical activity.

The devices are directed to helping the user improve the quality of the air they are breathing during certain activities. Typically, when people take a relaxing efficient breath, they inhale deeply and somewhat slowly from the nose filling their lungs before exhaling. In contract, during physical effort, people open their mouth, often gasping for air by breathing hard and quickly through the mouth. There is a tradeoff between quality and quantity between breathing through the nose versus the mouth. One can only get a small amount of air from the nose, so once the breath is elevated, people typically switch to the mouth. Mouth breathing quickly turns into shallow fast and heavy breaths. The present disclosure is directed to obtaining high-quality air, like that breathed through the nose, but with large volumes as that are breathed through the mouth.

The lungs operate effectively with specific qualities of air, specifically high moisture content and high temperatures. Without these qualities, the lungs dry out and become less efficient. As will be explained later, the nose enhances the quality of the air by at least four methods that are not available with mouth breathing.

There is a difference in air that enters lungs depending on if the air passes through the nose or the mouth. In addition, properties of exhaled air are measurable and informative about a user's breathing quality. To start, the nose purifies the air from pollutants with nose hairs, cilia, and mucus that captures dust and particulates. Air that passed through the nose (i.e. nasal cavities) is cleaner with fewer irritants as compared to mouth breathed air. The nasal cavities are lined with mucous membranes.

The nose has a large surface area, i.e. an internal surface of the nasal cavities and is connected to the trachea. These surface areas increase a temperature of the air inhaled. The temperature of air inhaled through the nose may be more than 4 degrees Celsius higher than air inhaled through the mouth. In the lungs, gas diffusion occurs through the alveoli or an alveolar wall with surfactants. Gas diffusion can be improved with an increase in temperature. Therefore, hotter inhaled air entering the lungs through the nose, has a larger efficiency in gas exchange with the blood, compared to the colder air inhaled through the mouth.

In addition, the nose increases the moisture of breathed air. The relative humidity (RH) of air inhaled through the nose, right before entering the lungs, is 100%. While air inhaled through the mouth is much closer to the ambient RH, which can be as low as 0% and is rarely above 50%. The present disclosure is directed to devices to increase one or both of temperature and humidity of air breathed in through the mouth.

A temperature of exhaled air (from nose and mouth) is in the range of 34 and 37 degrees Celsius with a relative humidity of 100%. It is noted that exhaled air that is leaving the body may have a larger water content than what was inhaled. This is more common with mouth breathing. Breathing air through the mouth results in a loss of moisture from the lungs as the air is processed and exhaled, a larger loss than that experienced by nasal breathing. As the dry air inhaled through the mouth reaches the lungs, it absorbs moisture from the lungs and impacts the performance of the surfactant on the lung walls. The moisture helps keep an effective, proper functioning shape of the lungs and alveoli, and the dissolved surfactants in the moisturized walls of the lungs helps speed up diffusion across the lung-blood membrane. Keeping the proper functioning shape of the lungs and the surfactants in a liquid form on the lung walls helps enhance gas diffusion.

An upper or top section of the lungs is first impacted by the drier air from the mouth. This dehydration of the top section of the lungs renders it less efficient. With more mouth breathing, more dry air is absorbing moisture from the lungs and the dryness of the lungs propagates through the lungs, constantly decreasing the lung efficiency and causing the breath to get shallower and quicker. At a threshold point, the lungs reach an unsustainable level where the person must stop the physical activity to help the lungs reset or rest. Managing the moisture in the lungs of a user can help with their performance, potentially increasing the threshold level of lunch capacity.

In addition, when breathing through the nose, a small amount of nitrous oxide (N2O) (such as approximately 100 parts per billion) is added before the inhaled air enters the lungs. Breathing through the mouth adds less than 20 parts per billion of nitrous oxide. An insignificant amount of nitrous oxide is recorded from trachea breathing in patients who have a tracheotomy surgery. The effects of nitrous oxide include improving the diffusion rate across the alveoli or lung-capillary interphase and is a vasodilator that facilitates profusion, increases blood-lung surface area, and helps in lowering both the heartrate and the blood pressure.

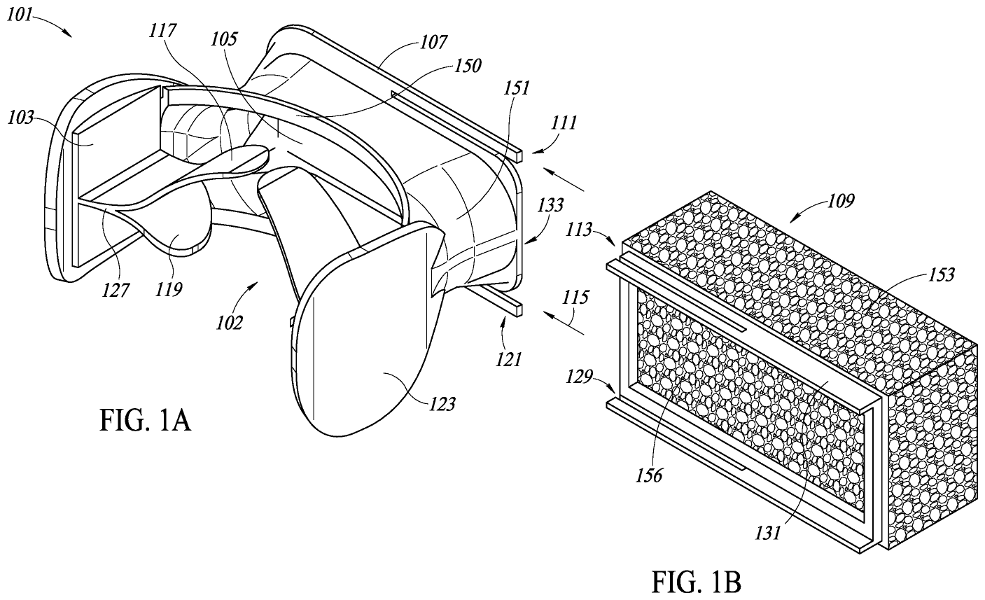

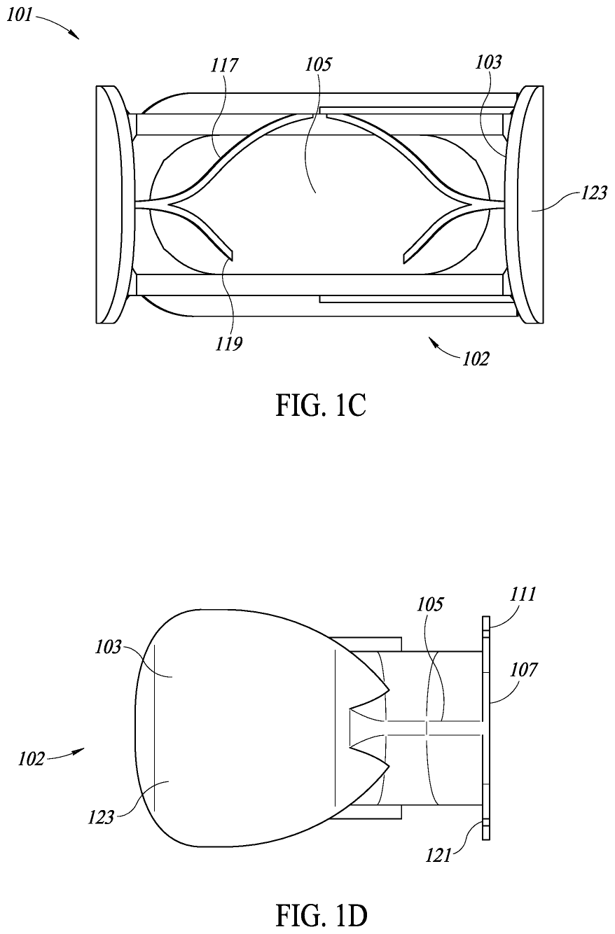

The devices of the present disclosure work to enable these beneficial aspects of nose breathing while breathing through the mouth. Various combinations of these aspects can be achieved with the passive and active breathing devices. The devices include an air filtration system coupled to a channel that is in fluid communication with a user's mouth. For example, FIG. 1A is directed to a perspective view of a passive embodiment of an inhaled air breathing device 101. The inhaled air breathing device 101 includes a mouthpiece 123 having at least one bite plate 103 extending from both sides of the inhaled breathing device 101 towards a central mouthpiece region 102. The mouthpiece 123 is coupled to an air filtration system 109 to allow the user to inhale air through a filter 153 to purify or clean the air. The air moves into a channel 105, which is a closed environment that can increase temperature and humidity of the inhaled air.

The bite plate 103 may have a single extension or may have multiple extensions that extend towards the central mouthpiece region 102, as will be described in further detail below. The mouthpiece 123 provides an easy, comfortable, and hands-free method for a user to keep the inhaled air breathing device 101 in their mouth, without difficulty, such as by biting the extensions.

In many exertive activities, athletes clinch their teeth. The extensions of this device will be held in place by the teeth of the user. The biteplate 103 of the mouthpiece 123 sits comfortably between the teeth of the user, with minimum jaw effort due to ergonomic shape and light weight of the device. A bite plate base 127 extends from the bite plate 103 and towards the central region. In this embodiment, the bite plate base 127 extends to an intersection where a top portion or section 117 separates from a bottom portion or section 119. The top section 117 and bottom section 119 are pulled away or extend away from each other. The shape and extension direction may be set by a mold during a manufacturing process. The presence of two extensions 117 and 119, is very useful in allowing the user to alternate between an open mouth bite and a closed mouth bite; biting on both extensions results in an open mouth which is useful for unimpeded large flowrates of air and this is the general practice in all mouthpieces. However, it is almost impossible to swallow your saliva with an open mouth which results in saliva buildup, discomfort, clogging, and possibly choking. Users may need to interrupt their activity to clear the saliva by removing the mouthpiece. Therefore, the design of these two extensions is made in such a way that the short extension 119 may be placed outside the bite/teeth, while the user bites on the long extension 117. Extension 117 are soft and elastically deformable, which allows it to contour into the bite profile. Notice that when a person has his mouth fully closed, the teeth of the bottom jaw sit inside the teeth of the top jaw and only then a comfortable swallow is possible. Therefore, each of the symmetric sections of 117 will deform to the space between the outside edge of the teeth of the bottom jaw and the inside edge of the teeth of the top jaw, while still having some portion of them contour the horizontal section between the top of the teeth of the bottom jaw and the mouth palate (roof of the mouth) for extra support. This is why the two parts of section 117 are not connected, since connecting the two ends will create tension across the segment and prevent the free contouring mentioned above. The benefit of this design also extends to protecting the teeth against grinding, and protects the jaw in contact sprots. Conventional protective mouthpieces cause an open mouth; while it protects the teeth by distributing the force of impact, it leaves the jaw vulnerable to excessive deviations which may cause knockouts, since the teeth of the bottom jaw are not fixed inside the teeth of the top jaw; however, this design allows to protect the jaw from such deviations by allowing the teeth to sit in their natural place.

Alternatively, a spring or torsional spring may be included between the sections 117, 119 or integrated in the sections. The springs may gently hold the top section 117 to the top teeth row and the bottom section 119 to the bottom teeth row, such that if the user's mouth is open during breathing, the top and bottom sections 117, 119 are pushing gently against the teeth and keeping the mouthpiece 123 and the breathing device 101 in place. This allows for breathing without clinched teeth. However, if the user chooses to close his mouth, for example, to swallow his saliva or is participating in a contact sport and wants to avoid having his mouth open during a collision, can do so by biting on the top and bottom sections 117, 119 and closing the gap. Said differently, the user may control the springs in a way that is comfortable and adaptable to the situation.

A channel or duct 105 is horizontally coupled to the mouthpiece 123. The duct 105 may be manufactured to be vertically inclined to further assist in preventing extra liquid or saliva from going through the duct 105. The channel 105 is in fluid communication with the central mouthpiece region 102. Air flows in and out through this channel 105. The channel includes an edge or lip 150 around which the user's lip can rest to keep the mouthpiece comfortably within the user's mouth. The channel extends from the edge 150 toward an attachment end or back plate 107 by a distance. Walls 151 of the channel are rigid in comparison to the extensions 117, 119 to be sufficiently strong to support the air filtration system 109.

The attachment end 107 is composed of a top insertion opening 111 and a bottom insertion opening 121. The top and bottom insertion openings 111, 121 of the attachment end 107 are receivable to an opposite mating device, such as a filter or foreign attachment.

In this embodiment, the top and bottom insertion openings 111, 121 of the attachment end 107 are configured to couple with the air filtration system 109, as shown in FIG. 1B. The air filtration system 109 has the filter 153 that may be composed of a hydrophobic material with large surface area, such as a foam, that is covered with a layer of hydrophilic salts. This family of filters is usually referred to as heat and moisture exchange filters (HMEF). The size of the foam used for the air filtration system and the density of its porosity 109 may be dependent on temperature and humidity, for example, colder weather requiring a larger foam size and smaller more numerous pores while warmer weather requiring a thinner foam size and larger less numerous pores. Some types of paper could be used as a substitute for the foam, and still generally known as an HMFE. The foam may have a limited life and be periodically replaced after exceeding a threshold usage time. The filter 153 may become discolored when it has reached its useful life. In these embodiments, the filter is left unbounded intentionally to allow user to see the discoloration when it happens, and not restrict the air in a linear direction but allow it to move in and out freely from all directions. This aspect significantly reduces the pressure loss making it much easier for the user to breathe the large amounts of air required during physical activity.

The air filtration system 109 works to absorb the heat and humidity of exhaled air during exhalation, because the exhaled air is warm and can have a 100% relative humidity. The air filtration system 109 then provides that heat and moisture to the inhaled air that is coming from the ambient air, which is dry and usually colder than the body's internal lung temperature. During inhalation, ambient air passes through the large surface area of the air filtration system 109 and encounters the wet and hot surface. As a result, the moisture and heat will be carried into the inhaled air. Thus, the air entering the mouth is much more favorable for the lungs, allowing the lungs to operate more efficiently.

The air filtration system 109 includes a frame 131 positioned on an inner perimeter that leaves a central area 156 open to be in fluid communication with the channel 105. The frame 131 includes a top insertion opening 113 and a bottom insertion opening 129. The top and bottom insertion openings 113, 129 of the frame 131 are sized and shaped to couple to the top and bottom insertion openings 111, 121 of the attachment end 107 by inserting or sliding in a first direction 115 towards an attachment back plate 133. In this embodiment, the frame 131 is a solid plastic or other suitable material that is light-weight and durable that creates the opening for the central area 156 of the filter 153. Other attachment techniques are envisioned, such as a snap and lock arrangement. —FIG. 1C is a view of the inhaled air breathing device 101 as described in FIG. 1A. The central mouthpiece region 102 is between the top and bottom sections 117, 119 that extend from the bite plate 103 towards the central mouthpiece region. The channel or duct 105 is positioned in line with the central mouthpiece region 102, and partly overlapped by the top and bottom sections 117, 119. This configuration of the inhaled air breathing device 101 enables a user to comfortably wear the device with the optimized duct having an efficient position for optimal airflow conditions. And finally, not depicted here, the mouthpiece can be directly connected or glued to the filter for a disposable version of this passive device.

As mentioned above, the mouthpiece 123 and bite plate 103 may be arranged in a variety of configurations. The bite plate 103 can be configured such that a single section extends from the bite plate 103, as opposed to two sections. The bite plate 103 may also be configured such that the two sections are of equal length and/or size and can be manipulated to fit the mouth and face of the user. The bite plate 103 and top and bottom sections 117, 119 may be composed of silicone, rubber, or other similar food grade quality deformable material. By slightly modifying this passive version of the device, the air inhaled breathing device 101 may be utilized as a protective mouthpiece, for example in physical contact sports such as American football, rugby, boxing, mixed martial arts, etc.

FIG. 1D is a side view of the inhaled air breathing device 101 as described in FIG. 1A. The mouthpiece 123 shows a slight curvature towards the central mouthpiece region 102. This enables the inhaled air breathing device 101 to remain fixed in the user's mouth, with assistance from the bite plate 103. The mouthpiece 123 is coupled to the channel or duct 105. In this embodiment, the channel or duct 105 is horizontally configured to enhance physical comfort in the respiratory system by ensuring that the lungs of the user will be much more comfortable using the air inhaled breathing device 101 as compared to breathing dry air from the mouth.

The channel or duct 105 couples the mouthpiece 123 to the attachment end 107. The configuration of the channel or duct 105 within the air inhaled breathing device 101 is to minimize the dead space of the channel or duct 105 that air flows in and out. The attachment end 107 is transverse to the channel or duct 105 and spans the width of the inhaled air breathing device 101. The attachment end 107 includes the top and bottom insertion openings 111, 121. The attachment end 107 may have top and bottom insertion openings 111, 121, or may have snap-on attachments, which will be described in further detail below. The attachment end 107 may also be modified or configured to fit another embodiment of the device.

In alternative embodiments, a nitrous oxide (N2O) canister (not shown) can be implemented in the passive and/or active versions of the air inhaled breathing device 101. For example, the nitrous oxide (N2O) canister may be connected to the bottom or top of the mouthpiece 123, or in the channel or duct 105 between the central mouthpiece region 102 and the air filtration system 109.

In this embodiment, the system does not extend too far from or beyond the profile of the mouth, which is very beneficial when the user is handling equipment like for example weight lifting, Olympic lifting, or CrossFit. This embodiment also minimizes the pressure loss and the deadspace. In alternative embodiments, the mouthpiece 123 may cover a mouth and/or nose, such as a mask covering the face, or may utilize straps that can be wrapped around the head or neck to hold the device in place. The mouthpiece 123 may also be held in position using an external fixture.

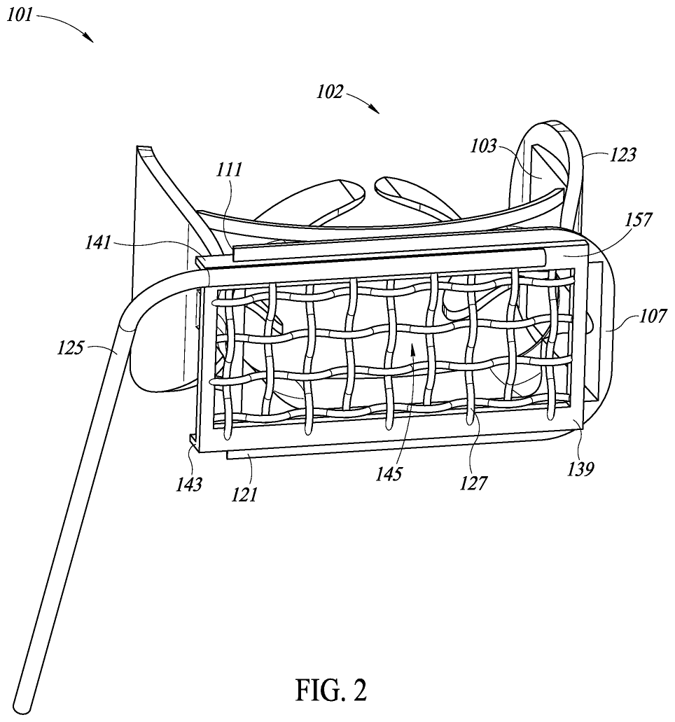

FIG. 2 is a perspective view of an embodiment of the air inhaled breathing device 101. The inhaled air breathing device 101 includes the mouthpiece features that are described in detail above. The central mouthpiece region 102 includes the bite plate 103 with the top and bottom sections 117, 119 extending from the mouthpiece 123 and bite plate 103. Alternative mouthpiece features may be combined with other aspects of this device and others in this disclosure.

In this embodiment, a heater device 139 is slidably received by the attachment end 107 of the air inhaled breathing device 101. The heater device 139 includes a top receivable opening 141 and a bottom receivable opening 143. The top and bottom receivable openings 141, 143 are slid into the top and bottom interposable openings 111, 121 of the attachment end 107. This form of attachment may be modified to include a snap-on attachment or other form of attaching the filter to the device.

The heater device 139 includes a plurality of openings 145 that intersect a plurality of thermally conductive bars or wires 127. The plurality of openings and thermally conductive bars 145, 127 extend and span the entirety of the heater filtration system 139 to form a grid or array. The thermally conductive bars 127 are covered with a protective or dielectric coating to prevent injury. The conductive bars are powered from a remote power device through connection 125. This connection may be at any location of the heater device 139. A small, replaceable battery may be coupled to the heater as an alternative to the connection 125.

The heater warms a temperature of the air being inhaled as the user breathes through their mouth. A filter may be incorporated into the heater matrix such as between the conductive bars and the channel 105. In addition, a water atomizer, which creates water vapor from a container of water resulting in moisturizing the inhaled air, can be integrated into this active device, such as through the tubing of the connection 125 from a remote container. For example, the connection 125 may include a water channel and a wire for power within this connection 125. These two steps can be in series, where the inhaled air first passes through the heating section and then passes through the moisturizing section, such that the moisture is provided just before the channel. Alternatively, the moisture can be in parallel where moisture is inserted from the top down onto the conductive bars such as through a plurality of nozzles in the upper part of a frame 157. The stream of air is heated and mixed with a stream of moisturized air before being inhaled by the user.

This active version of the inhaled air breathing device 101 requires an input of power for the thermally conductive bars 127. The power source or battery (not shown) for the inhaled air breathing device 101 may be integrated in a variety of configurations, such as integrated into the device close to the user's mouth, or as shown in FIG. 2, connected via a power cable/connection 125 to a distal location from the user's mouth. The benefit of this embodiment is that it can provide the optimal conditions of the air, exact temperature, and exact humidity as opposed to the passive form which may recover only some of the heat and moisture.

The inhaled air breathing device 101 may be manually controlled to allow the user to decide when and how much power they want to extend into heating the air and evaporating the water. The user may choose to apply an automatic setting where the device is activated precisely when the user is inhaling, using pressure or flow sensors (not shown), and calculates the amount of power to put into the heaters and evaporators based on the atmospheric conditions and breathing rate of the user. The sensing of the atmospheric conditions can be done with sensors on the device itself, or by connecting the device to an application (app) on the mobile phone that can feed the information from a weather application or other external sensor.

With sensors, an algorithm can be utilized to determine the user's optimal air conditions, which can vary depending on the ambient conditions and/or vary from one user to the other and/or from one activity to another.

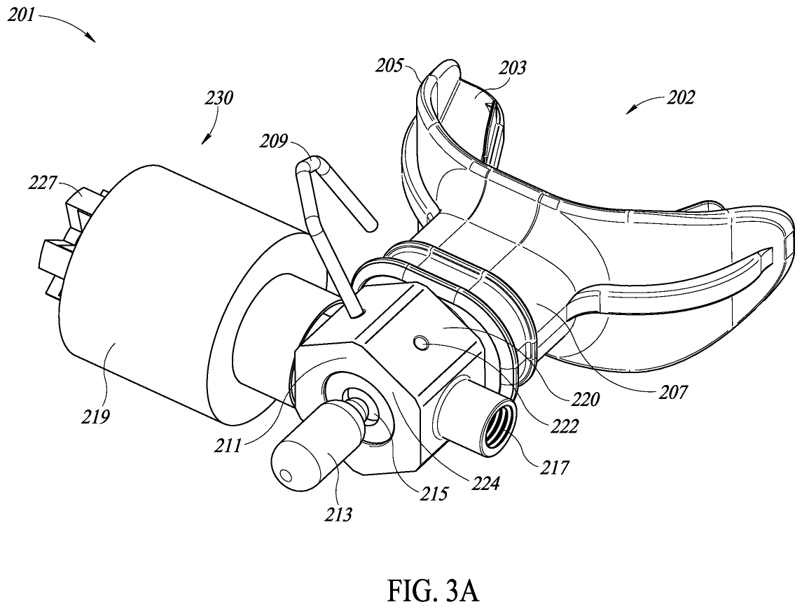

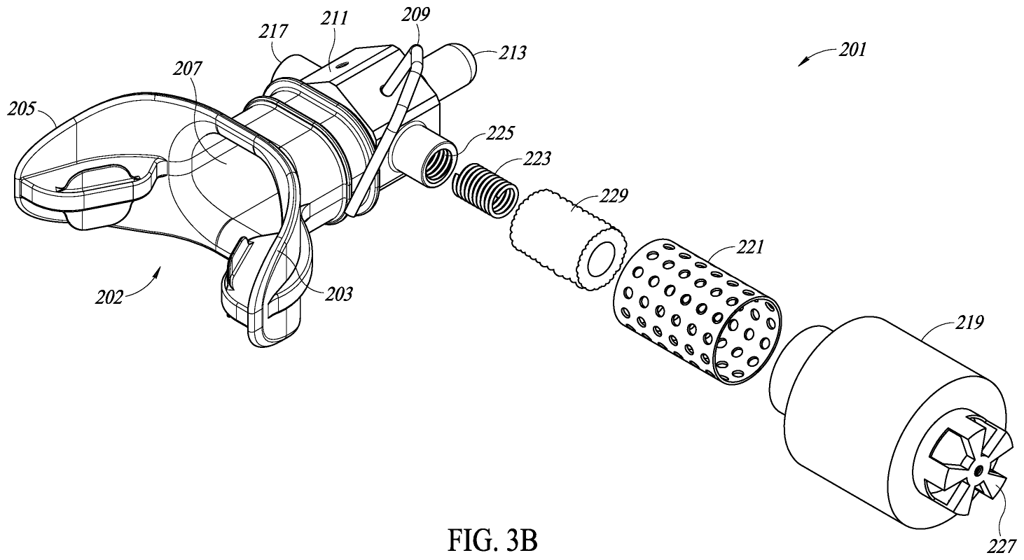

FIG. 3A is a perspective view of an embodiment of an active inhaled air breathing device 201 having a moisture and temperature adding system coupled to a mouthpiece. In this embodiment, the inhaled air breathing device 201 includes a central mouthpiece region 202 within a mouthpiece 205 having at least one bite plate 203 extending from both sides of the mouthpiece towards the central mouthpiece region 202. In this embodiment, the bite plate 203 has a single extension that can be gripped by the user's mouth or teeth to position and hold the device. As mentioned above, the bite plate 203 may encompass a variety of modifications tailored to the individual user. The bite plate 203 can rest between the top and lower row of teeth, or may be positioned in the user's mouth as deemed fit by the individual user.

A channel or duct 207 extends from the mouthpiece 205 and is aligned with the central mouthpiece region 202. The duct 207 couples the mouthpiece 205 to an air filtration base 211 that is hexagonally shaped in FIG. 3A, but can have a variety of shapes. The air filtration base 211 may be a valve, base, drum, or other similar structure that securely attaches the channel to user selected additional components, like nitrous oxide canisters 213 or moisture generation systems 230. The air filtration base 211 is centrally located opposite to and at a distal point from the central mouthpiece region 202 via the channel or duct 207. The air filtration base 211 is the section of the device that mixes all the incoming streams together to create a homogenous mixture before entering the lungs. The air filtration base 211 is designed as such to cause minimum pressure loss. The air filtration base 211 includes a plurality of sides or surfaces 220, all having variable types of functionality. Each side 220 faces externally from an interior chamber of the base 211. Each side 220 may be have an opening 222 for an attachment. Each opening may have a one way valve into the chamber to prevent the back flow of air or other materials.

A surface 224 of the air filtration base 211 that is transverse to the sides 220 includes a valve to receive a nitrous oxide (N2O) canister or cartridge 213. The valve or diffusion membrane 215 allows a controlled amount of nitrous oxide 213 to diffuse from a region of high nitrous oxide concentration, the canister or cartridge side, to the region of low nitrous oxide concentration, the interior chamber and the channel. The addition of nitrous oxide may not be by means of a purified nitrous oxide canister, but may be applied by numerous other methods of producing nitrous oxide on site, such as several chemical reactions.

Another side of the air filtration base 211 is an unused attachment region 217 that may include threads or other mechanism for coupling additional components. For example, an air heating coil or mesh may be integrated in this region. The air heating coil or mesh 217 could heat a portion of the inhaled air entering from this side. In this embodiment, the air heating section is performed parallel to the other processes described above. However, this process can be done in series prior to the moisturizing section, or simultaneously with the evaporation section.

These unused regions can be coupled to other canisters, like asthma inhaler compounds, e.g. Albuterol, or oxygen, specifically for high altitude adventures. An oxygen canister can help boost that concentration to the normal levels that one experiences at sea level, or even higher.

A moisture generation systems 230 extends from another side of the base 211 and includes a water tank 219. The water tank 219 is a container of water or other suitable fluid to provide humidify to the inhaled air. The material may be made of glass, or Plexiglas, or plastic. This material is suitable because it allows the user to see how much water is in the water tank 219, while also operating as a good heat insulator. Other materials and elements, which will be discussed in further detail below, are housed within the moisture generation systems 230 and coupled together with a fastener 227. The fastener 227 may be a wing nut, bolt, or other similar fastener used to secure and contain the materials within the moisture generation systems 230.

Located on an alternative side of the air filtration base 211 is a power cable 209. Similar to as described above in other embodiments, the power cable 211 connects the coils to a power supply element (not shown), which may be a battery or power source attached to the device, or another power source housed away or detached from the device.

FIG. 3B is an exploded view of the inhaled air breathing device of FIG. 3A. Particularly, FIG. 3B shows an exploded detailed view of the materials and elements housed within the moisture generation systems 230. Opposite from the air heating coil or mesh 217 is an evaporator coil or element 223 that is coupled to a housing 225 extending the air filtration base 211. The housing 225 may be an integral part of the base 211 or may be a user selectable attachment. A barrier mesh 221 is inside of the water tank 219 to filter the water into a wick 229 and regulate a flow of the water. The barrier mesh 221 is a thin, perforated mesh that restrains water from falling inwards into the center of the water tank 219. This is achieved by the water surface tension on edges of the small perforations.

A wick or absorbing material 229 is placed inside of the barrier mesh 221 and surrounds the evaporator coil or element 223. The wick may be cotton or silk or other suitable material. In an alternative embodiment, the wick can be a sintered metal. Small metal pebbles that are fused together can create a wick. These metal options have an advantage of never burning and thus are able to handle much higher temperatures and power output of the evaporator coil.

The wick or absorbing material 229 is fast absorbing and helps transport the water quickly from the perforation of the barrier mesh 221 to the surface of the evaporator coil 223 without drowning the coil or the air passage inside the coil. The water tank 219 encapsulates or houses all the elements mentioned in detail above, and are securely held in place by the fastener 227. The moisture generation systems 230 may be inhale activated to draw the moisture into the channel in the mouth piece. The heater coil 223 can add temperature while also adding moisture from the water tank and other elements.

There are several benefits of this method of moisturizing, such as the fast stream of inhaled air passes directly within the region of evaporation, leading to efficient moisturizing of the air and homogenous mixture of humidity inside the air. Another benefit is that only the amount of water required to be evaporated is in contact with the heating element, which provides an almost immediate on-command start and shut-off of the evaporation process. There are also other methods to humidify that are not described, such as boing the water with a coil placed inside, however this process doesn't hold the same advantages as this particular method.

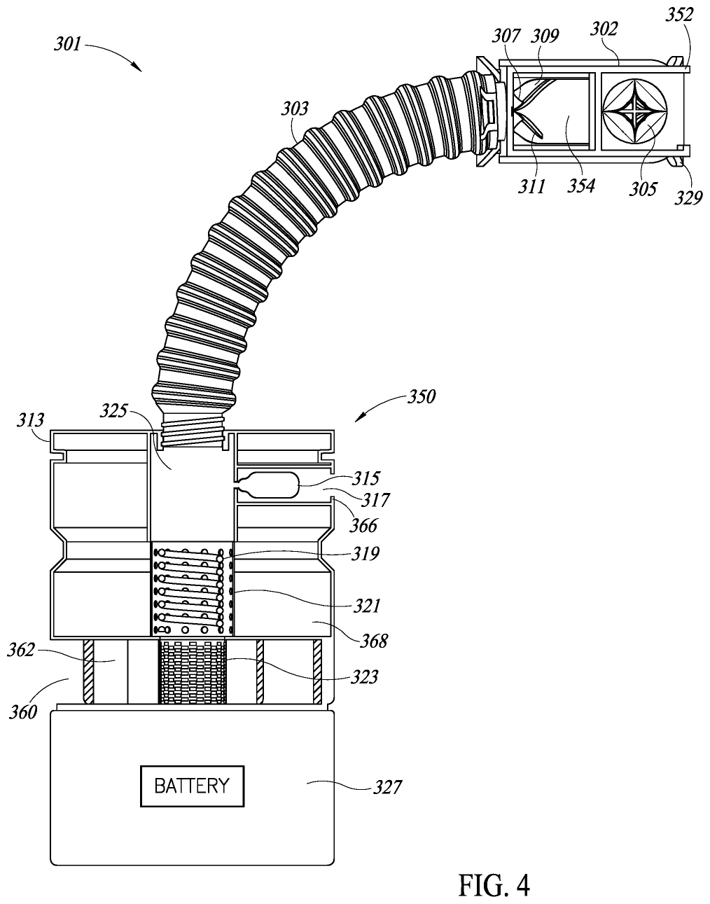

FIG. 4 shows an embodiment of an active inhaled air breathing system 301 that includes a larger moisture and temperature generating system 350 coupled to a mouthpiece 329 with tubing 303. The moisture and temperature generating system 350 is shown in cross-section to provide details about the features. The inhaled air breathing system 301 includes an inhaled air breathing device 302 that includes a mouthpiece 329 having at least one bite plate (not shown). The mouthpiece is coupled to a removable or detachable coupling device 352 that includes a rigid frame. The coupling device is attached in close proximity to the mouthpiece 329 and includes a first one-way valve 305 to allow exhaled air to exit a chamber 354 formed in this closed system when in use. In this embodiment, the one-way valve 305 forces the exhaled air to exit the device very close to the mouth region, without passing through the rest of the system, which is blocked by a second one-way valve 307. The air inhaled on the following breath will not have any portion of the exhaled air from the previous breath, thus eliminating what is referred to as “deadspace.”

The frame includes a top portion or section 309 and a bottom portion or section 311 that are coupled to the mouthpiece. The top and bottom portions 309, 311 are closer to the second one-way valve 307 that is coupled to the tubing 303. The tubing or duct 303 extends from the inhaled air breathing device 302 mouthpiece 329 to the moisture and temperature generating system 350. The moisture and temperature generating system 350 includes an air filtration base system 313 that allows air to move into the system 350 through a plurality of openings 360. Both the first and second one-way valves may include a filter to remove particulates from the air.

The plurality of openings 360 are separated by supports or extensions 362. A fine, barrier mesh filter 323 is centrally positioned within the plurality of openings 360. The extensions 362 may be positioned around a perimeter of a chamber around the filter 323 or may be interspersed throughout this area.

The air filtration base system 313 includes a chamber 325 located at an end point of the duct 303. A nitrous oxide (N2O) canister or cartridge 315 is inserted through an opening or void 317 in a side or wall 366 of the chamber 325. The nitrous oxide canister 315 may be inserted or screwed into this embodiment. The nitrous oxide canister 315 may utilize a cover, which would contain the canister or cartridge and would be screwed to the device. The opening 317 allows the canister to be removably attached to the internal humidified air, valve, or diffusion membrane. This allows for a precise amount of nitrous oxide to flow into the chamber 325. The chamber 325 is a closed system with the user's mouth during use.

A heating element or coil 319 is located in the central region of the air filtration base system 313. The heating element 319 is surrounded by a water wick material (not shown for drawing clarity), similar description as that of 229 above, which is in turn surrounded by a barrier mesh 321, similar to the description of 221 above. The water wick mesh 321 is connected to a container or reservoir 368 of water that surrounds the water wick material, the barrier mesh 321 and heating element 319. The water wick material 390 absorbs the water from the water reservoir across the openings of the water barrier 321, such as by surface tension. As the inhaled air flows through the air filtration base system 313 from the openings 360, it is first preheated by the heater mesh 323, then at the location of the inner opening of the evaporator coil 319 it can carry the water vapor formed by evaporating the water from the wick material by the heat of the evaporator coil. This is a series configuration of preheat, through the filter 323, followed by evaporation, with the coil 319, the water wick material, and the mesh 321, which allows more control on exactly how much energy you need to put into air temperature rise and how much energy you need to put into a precise amount of water vapor formation. Alternatively, the two aspects could be combined in a single step but does not give you the ability to optimize the heating and humidification characteristics.

The active form of the devise is made in an ergonomic design, like a water bottle, to make it easy for the user to handle and to stew in where a water bottle would normally go.

A power source or battery 327 is positioned at a lower level of the system 350. Similar to other embodiments, the power source 327 helps power the heating elements, but may also power flow, temperature, and/or humidity sensors. By utilizing pressure or flow sensors, the device is capable of calculating the amount of power to put into the heaters and evaporators based on atmospheric conditions and breathing rate of the user. For example, a sensor may be positioned in the chamber 325 to determine temperature and humidity. The sensor may be coupled to a processor or application specific integrated circuit in the battery area 327 that is configured to receive and respond to the measured temperature and humidity. The processor may increase a temperature in response to the temperature being less than a threshold.

In an alternative embodiment, nitrous oxide can be by dissolving nitrous oxide in the water that is to be evaporated by the atomizer or moisture generation system. So that when the water is evaporated, the dissolved nitrous oxide will also be carried by the inhaled air stream.

Another alternative includes using hydrogen peroxide (H2O2) instead of water, or an optimized mixture of the two. H2O2 decomposes completely into H2O and O2, especially with the addition of heat. Therefore, we can evaporate H2O2 into the inhaled stream which, besides increasing the water content, increases the O2 content of air. This will increase the partial pressure of O2 in the lungs and increases both the rate and the amount of O2 diffused into the lungs. Alternatively, this can also be achieved by including a liquid O2 canister similar to that utilized for the nitrous oxide.

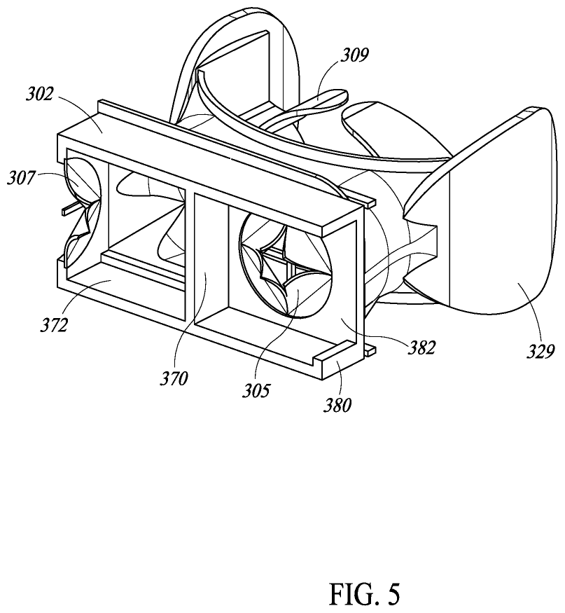

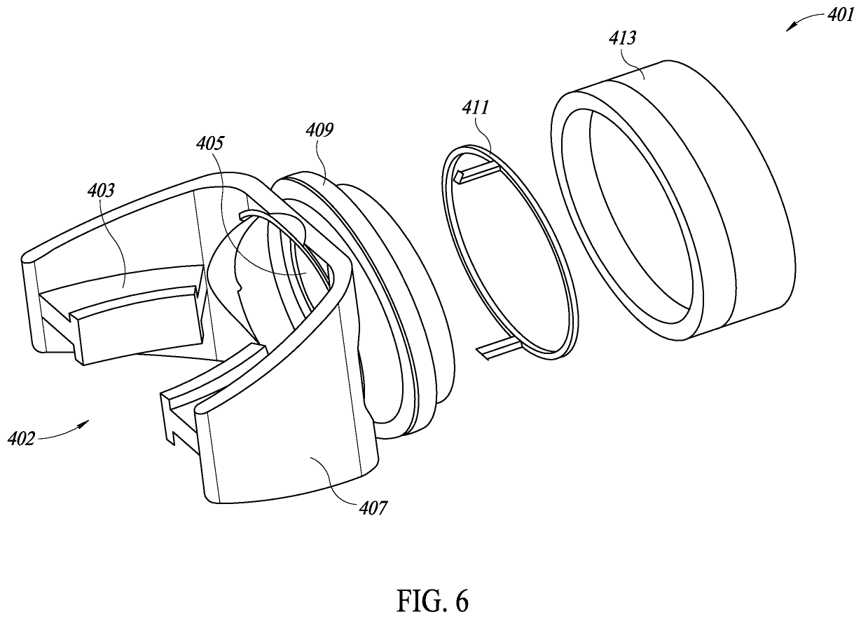

FIG. 5 shows a stand-alone perspective view of the inhaled air breathing device 302 of FIG. 4. The first and second one-way valves 307, 305 are more easily viewed. A wall 370 separates an in-flow chamber 372 from the first one-way valve 305 where air escapes or is vented out. A side 380 includes an opening 382 in the frame that allows air to flow out if a cover (not shown) is included on an external face (away from the user). The chamber 372 is closed or otherwise sealed so that the inhaled air flows in from the tubing and through the one-way valve 307. This device is washable and reusable.

When a user inhales using the inhaled air filtration system 301, flow sensors may then activate the heating system automatically and deactivate the system during exhale. The flow sensor(s) may be installed within the inhaled air breathing device 302 by the second one-way valve 307, or in a separate location if needed.

FIG. 6 shows an exploded view of an alternative passive embodiment of an inhaled air breathing device 401. A mouthpiece 407 having at least one bite plate 403 extends towards a central mouthpiece region 402. The bite plate 403 in this embodiment has a single extension, capable of the top and bottom teeth of a user to grip the bite plate 403 and hold the device in place. A channel or duct 405 is horizontally coupled to the mouthpiece 407. The duct 405 is aligned to provide adequate inhaling and exhaling of air into the lungs.

At an opposite end of the mouthpiece 407 is a heat and moisture exchange filter 413. This heat and moisture exchange filter 413 is a made of a sponge-like material that has many small passages that maximize the surface are. It works to absorb the heat and humidity of exhale air during exhalation and then gives back that heat and moisture to the inhaled air, similar to air filters described in other embodiments mentioned above. The duct 405 couples the mouthpiece 407 to the heat and moisture exchange filter 413 by a saliva trap 409 and snap-on attachment 411.

During an activity, a person may be in an orientation that forces saliva from the user's mouth to go down the filter 413. To prevent that saliva from reaching the filter 413 and clogging it up, the saliva trap 409 is inserted to contain and store the extra liquid. The saliva trap 409 is coupled to the duct 405 and the mouthpiece 407, formed like a container that holds the excess liquid, thus preventing the liquid from reaching the face of the heat and moisture exchange filter 413. The saliva trap 409 may be made as a single part along with the heat and moisture exchange filter 413, such as a heat and moisture combined saliva trap filter (not shown). The filter would have a longer base and function as an extended tunnel. This would be made to allow the saliva to be absorbed by the spongy tunnel, preventing the excess liquid and/or saliva from hitting the face and clogging the filter.

These devices allow the respiratory system, the circulatory system, and the muscular system to operate at optimal conditions and provide superior performance, as well as eliminate or minimize pains and soreness. Using these devices can reduce the recovery time from strenuous activities.

These devises can be used even if no hard physical activity present, such as playing in the park or walking around in a cold environment, which causes stress on the lungs and reduces performance capabilities. With increased physical output the device's benefits increase.

These devices can be used by military and army personals who perform hard physical activity and in harsh environments like dry dessert or extreme cold. Snow and winter sports can benefit from this devise due to the harsh conditions. People who work in dry conditions may see improved performance and feel healthier using this device. These devices can be attached/appended to breathing systems of the individuals that need onboard air supply, like scuba divers, high altitude climbers, etc. Also, with small modifications to the mouthpiece to enhance the protective aspects for the user, like a protective mouthpiece, the device can be used by athletes in contact sports like American football, rugby, boxing, MMA etc.

These devices provide physical comfort in respiratory system where the lungs of the user will be much more comfortable using this device as compared to breathing dry air from the mouth. This benefit is noticeable within merely a minute of using this device. No heavy breathing, no scarring, no itching or burning sensation.

These devices enhance respiratory system efficiency, increase endurance, and enhanced respiratory system recovery. The endurance of the user will be amplified, as the lungs work efficiently without limitation. As such, the physical activity can be performed for a much longer period with much more comfort and ease. Moreover, since this devise helps keeps the lungs hydrated and lubricated, it significantly reduces the possibility of having exercise induced asthma, a condition that faces anyone who pushes their respiratory system to the limit, which may sometimes require days of recovery while experiencing pain and coughing and shortness of breath.

Cardiac system fatigue may be reduced and heartrate controlled by using these devices. This will reduce suffering in the lungs from inefficiencies due to dryness as a person tries to breathe more air through the mouth. The dried areas of the lungs and alveoli lack the ability to properly exchange gasses with the blood arterial capillaries. As a result, the carbon dioxide (CO2) concentration in the blood plasma increases, which decreases the Ph of the blood. When the brain senses the lower Ph of the blood, it signals the heart to beat faster. The heart is aiming to pass more blood through the lung-arterial capillaries interface to get rid of the extra CO2. However, the blood CO2 keeps rising with continued exercise due to the inefficient lung surfaces, causing the heartrate to continuously increase until cardiac fatigue occurs, where the human is forced to dial down his activity or even stop it completely. With these devices, the lungs stay properly lubricated, oxygen is transported to the blood and CO2 is taken from the blood more efficiently, then the heart does not have to work overtime to compensate for the lung inefficiencies. The user will feel less fatigued, and with much less cardiac overload.

These devices can support a muscular system with increased power output, elimination of soreness, and faster recovery, which are all impacted by an amount of oxygen available for the muscles. Without oxygen, the body reverts to lactic acid cycle (or anaerobic cycle) which, unlike the oxygen rich cycle (aerobic cycle), breaks down carbohydrates in the absence of oxygen. The lactic acid cycle is less efficient in producing energy (produces less energy units (ATP) for every carbohydrate burned), but produces lactic acid as a byproduct. The lactic acid buildup causes the muscles to tire, ache, and burn, causing the performance to drop or cramps to occur. The devices improve oxygen transport from the lungs to the blood and thus to the muscle tissues. This can delay or avoid the lactic acid cycle. The muscles will continue to operate efficiently for much longer periods, with high-power output, without aches or pains. This may also reduce the body's recovery time.

Heartrate control and elimination of uncontrollable shallow breathing may be achieved with these devices. This can prevent the Herring-Breuer reflex, where a person inhales sharply with the lungs being over stretched. This over stretching is sensed by receptors in the alveoli that triggers the Herring-Breuer reflex, causing the lungs to operate under short shallow and quick breaths and the heart increases, possibly to sinus arrhythmia. While normal, it is a sign to end the physical activity. This device allows the user to inhale where the lungs may no longer be the limiting factor. The devices can limit the impact of a more aggressive inhale.

A user may have an increased willingness to start and/or extend exercising, which can support weight loss, by reducing the pain and aches and exhaustion that the muscular system, the respiratory system, and the circulatory system they have previously experienced. The devices can reduce water loss from exercise via the respiratory system, reducing the need to drink water. The devices reduce the feeling of the need to drink water as the mouth and lungs will not be a dry. This dry mouth and lungs create a thirst sensation even if water levels in the body are adequate. So, if the athlete doesn't drink, they feel thirsty which is both uncomfortable and reduces focus on the task at hand. Moreover, the brain signals to reduce the body's work output when it senses thirst whether the body is dehydrated or not. On the other hand, if the user does drink water, especially when not needed by the body at point in time, it will be an uncomfortable feeling in the stomach and possibly creating cramps and spasms, not to mention that it may increase the body weight which is a crucial thing in a professional and competition environment. That is why we sometimes see athletes rinse their mouth then spit most of the water out, and they may do this more than once consecutively iterating between the proper portions of water needed by the body vs water to just eliminate the physiological sensation of thirst due to dryness. This requires huge levels of experience by the athlete and even then, it is a time-wasting procedure.

As noted above, the devices can be adjusted for animals. For example, horse races, horse hurdles, dog races, dog hurdles, camel racing, sled dogs, etc. The device can also be modified for the health of any pet that is performing a healthy dose of its daily activity.

Embodiments of the present disclosure include a bite plate that allows for both an open mouth and a closed mouth options for the bite. There is a channel coupled to the bite plate and an attachment end spaced from the bite plate by the channel, the attachment end including an opening in fluid communication with the channel. There is an air filtration system removably coupled to the attachment end. This system does not extend too far from or beyond the profile of the user's mouth.

The attachment end includes a frame having a first air filtration coupling track and a second air filtration coupling track. The opening is between the first air filtration coupling track and the second air filtration coupling track. The first air filtration coupling track and the second air filtration coupling track open from a first side of the attachment end and extend towards a central region of the attachment end. The air filtration system includes a first coupling track and second coupling track that are slidably received by the first and second air filtration coupling track on the attachment end.

The bite plate is in a chamber, the bite plate has first upper extensions and second lower extensions, the first upper extensions extending further in the chamber than second lower extensions. Alternatively, the bite plate is in a chamber, the bite plate includes a single extension that extends in the chamber. The air filtration system includes a frame that is configured to be coupled to the attachment end and the air filtration system includes a filter coupled to the frame. Alternatively, the air filtration system includes a heat exchange filter having a plurality of conductive bars.

Another embodiment includes a bite plate; a duct attached to the bite plate; a removable air exchange attachment detachably coupled to the duct; a moisture tank coupled to the air exchange attachment; and a heating element coupled to the air exchange attachment. A nitrous oxide canister can be coupled to the device. The moisture tank includes a heating coil and a mesh. The removable air exchange attachment includes a plurality of openings. A flow sensor, a temperature sensor, and a humidity sensor may be included.

The present disclosure is also directed to a system that includes a breathing device to be held in a user's mouth having a first end and a second end; a duct coupled to the second end of the breathing device; and an air management system coupled to the breathing device by the duct, the air management system having a first one-way valve. The air management system includes a second one-way valve, the first one-way valve configured to allow inhaled air into the breathing device and the second one-way valve configured to allow exhaled air out of the breathing device. A heating tank coupled to the duct. A nitrous oxide cartridge valve in the air management system.

The various embodiments described above can be combined to provide further embodiments. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A device, comprising:
a bite plate;
a channel coupled to the bite plate;
an attachment end spaced from the bite plate by the channel, the attachment end including an opening in fluid communication with the channel;
an air filtration system removably coupled to the attachment end.

2. The device of claim 1 where in the attachment end includes a frame having a first air filtration coupling track and a second air filtration coupling track.

3. The device of claim 2 where in the opening is between the first air filtration coupling track and the second air filtration coupling track.

4. The device of claim 3 wherein the first air filtration coupling track and the second air filtration coupling track open from a first side of the attachment end and extend towards a central region of the attachment end.

5. The device of claim 2 wherein the air filtration system includes a first coupling track and second coupling track that are slidably received by the first and second air filtration coupling track on the attachment end.

6. The device of claim 1 wherein the bite plate is in a chamber, the bite plate has first upper extensions and second lower extensions, the first upper extensions extending further in the chamber than second lower extensions.

7. The device of claim 1 wherein the bite plate is in a chamber, the bite plate includes a single extension that extends in the chamber.

8. The device of claim 1 wherein the air filtration system includes a frame that is configured to be coupled to the attachment end and the air filtration system includes a filter coupled to the frame.

9. The device of claim 1 wherein the air filtration system includes a heat exchange filter having a plurality of conductive bars.

10. A device comprising:
a bite plate;
a duct attached to the bite plate;
a removable air exchange attachment detachably coupled to the duct;
a moisture tank coupled to the air exchange attachment; and
a heating element coupled to the air exchange attachment.

11. The device of claim 10 wherein the moisture tank includes a heating coil and a mesh.

12. The device of claim 10 wherein the removable air exchange attachment includes a plurality of openings.

13. The device of claim 10 further comprising a flow sensor, a temperature sensor, and a humidity sensor.

14. A system comprising:
a breathing device to be held in a user's mouth having a first end and a second end;
a duct coupled to the second end of the breathing device; and
a air management system coupled to the breathing device by the duct, the air management system having a first one-way valve.

15. The system of claim 14 wherein the air management system includes a second one-way valve, the first one-way valve configured to allow inhaled air into the breathing device and the second one-way valve configured to allow exhaled air out of the breathing device.

16. The system of claim 14 further comprising a heating tank coupled to the duct.

17. The system of 14 comprising a nitrous oxide cartridge valve in the air management system.
